(19) United States
(12) Patent Application Publication
(10) Pub. No.: US 2007/0154568 A1
(43) Pub. Date: Jul. 5, 2007

(54) Clay Purification Method

(75) Inventor: Denise FAYOLLE, Velleron (FR)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Societe de Conseils de Recherches et D'Applications Scientifiques (S.C.R.A.S.), Paris (FR)

(21) Appl. No.: 11/686,233

(22) Filed: Mar. 14, 2007

(30) Foreign Application Priority Data
Sep. 17, 2004 (FR) 0409835

(51) Int. Cl. A61K 33/12

(52) U.S. Cl. 424/683

(57) ABSTRACT

The invention relates to a method for the purification of a clay that is intended for therapeutic use.

The present invention relates to a process for the purification of a clay for therapeutic use. This clay can be a clay of smettie, beidellitic montmorillonite or attapulgite type.

Clay is a natural product known for its absorbent and adsorbent power. Thus the clay of smettie type, marketed under the trade-mark Smecta®, is used for example in the symptomatic treatment of the pain linked to oesophago-gastro-duodenal diseases and colics and acute and chronic diarrhea.

The raw material generally only contains approximately 30 to 60% of active ingredient, it is therefore indispensable to purify it in order to obtain a pure clay in the pharmaceutical sense.

A process for the preparation of a clay for therapeutic use is described in the patent FR 2346017. The latter comprises the following main stages: a grinding stage, putting into suspension in water, a chemical treatment of the suspension obtained with an acid until stabilization at pH 2-3, a physical treatment in hydrocyclones and finally drying.

The Applicant has found that the chemical treatment with stabilization at pH 2-3 as described in the patent FR 2346017 was not necessary and that the chemical treatment could be carried out at a less acid pH.

Therefore a subject of the present invention is a process for the purification of a clay for therapeutic use, the process comprising the following successive stages:
1) a mechanical treatment of the raw clay; this is a course grinding of the raw material in order to reduce the chunks to dimensions which are acceptable for the rest of the process and to eliminate undesirable hard material such as gravel;
2) putting into suspension in water in order to allow purification of the clay by wet route followed by a granulometric treatment which allows the clay to be reduced to particles which are sufficiently fine to be distinguished from foreign particles still present but of larger dimensions;
3) a chemical treatment in order to eliminate the limestone which is the only residual impurity in the clay in suspension;
4) a physical treatment including decantation/centrifugation, which allows a progressive selection of the raw clay; and finally
5) a heat treatment which allows the recovery of a pure and sterilized clay,
   a process characterized in that the chemical treatment is carried out by adding a strong acid for stabilization at a pH of the order of 3-5.

The clay thus obtained is a pure clay in the pharmaceutical sense.

Preferably, the process of the invention is carried out for clays containing alkaline-earth carbonates, and more particularly alkaline-earth carbonates selected from K₂CO₃, Na₂CO₃, MgCO₃ and CaCO₃.

Preferably, the chemical treatment of a process according to the present invention is carried out at a pH of the order of 3.5-4.5, and very preferentially at a pH of the order of 4-4.5.

Preferably also, the chemical treatment of a process according to the invention is carried out by adding a strong mineral acid, and very preferentially by adding hydrochloric acid.

Preferably also, the chemical treatment of a process according to the invention is carried out by adding an acid in a ratio (quantity of acid/quantity of raw material) comprised between 0.010 and 0.020.

A process according to the present invention therefore has the advantage of using less acid which allows the limiting and/or elimination of the release of acids and acid vapours. Moreover, this reduction in the consumption of acid which is capable not only of degrading the clay but also the apparatuses used, also has advantages both for the final product but also in terms of cost.

All the technical and scientific terms used in the present text have the meaning known to a person skilled in the art.

### EXPERIMENTAL PART

After extraction of the raw material (approximately 1 kg) and the mechanical treatment, the clay is put into suspension then subjected to a granulometric treatment in order to reduce the clay to sufficiently fine particles. The suspension (20% in water) is subjected to stirring and 0.010 to 0.020 kg of 36% hydrochloric acid is then added in order to obtain a pH value of the order of 3-5. Initially, the acid is added in excess in order to be sure to eliminate all the carbonates which could be present. Complete neutralization of the carbonates is carried out by monitoring the pH value.

After physical treatment using hydrocyclones and returning to a solid phase, the paste is dried in order to thus obtain a pure clay in the pharmaceutical sense.

1. Process for the purification of a clay for therapeutic use, the process comprising the following successive steps:
1) mechanically treating a raw clay;
2) suspending the clay in water followed by granulometrically treating the clay;
3) chemically treating the clay in order to eliminate any non-argillaceous minerals;
4) physically treating the clay; and
5) heat treating the clay and recovering a pure and sterilized clay,
wherein the chemical treatment comprises adding a strong acid for stabilization at a pH of 3-5.

2. Process according to claim 1, wherein the clay includes alkaline-earth carbonates.

3. Process of claim 1, wherein the clay comprises alkaline-earth carbonates including K₂CO₃, Na₂CO₃, MgCO₃ or CaCO₃.

4. Process of claim 1, wherein the chemical treatment is carried out at a pH of 3.5-4.5.

5. Process of claim 1, wherein the chemical treatment is carried out at a pH of 4-4.5.

6. Process of claim 1, wherein the chemical treatment comprises adding a strong mineral acid.

7. Process of claim 1, wherein the chemical treatment comprises adding hydrochloric acid.

8. Process of claim 1, wherein the ratio (quantity of acid/quantity of raw clay) is between 0.010 and 0.020.
